(19) United States
(12) Patent Application Publication
(10) Pub. No.: US 2024/0041731 A1
(43) Pub. Date: Feb. 8, 2024

(54) TWO-PHASE COSMETIC COMPOSITION COMPRISING AN AMINO ACID AND TWO SPECIFIC ACIDS

(71) Applicant: L’OREAL, Paris (FR)

(72) Inventors: Céline DEBOVE, Chevilly Larue (FR); Camille POSTIC, Chevilly Larue (FR)

(21) Appl. No.: 18/255,964

(22) PCT Filed: Dec. 16, 2021
(86) PCT No.: PCT/EP2021/086106

§ 371 Date: Jun. 5, 2023

(30) Foreign Application Priority Data
Dec. 17, 2020 (FR) FR2013459

(51) Int. Cl. A61K 8/44; A61K 8/365; A61K 8/73; A61Q 1/14

(52) U.S. Cl. CPC A61K 8/44; A61K 8/365; A61K 8/73; A61Q 1/14

(57) ABSTRACT

The present invention relates to a cosmetic composition consisting of an aqueous phase and an oily phase which are separate, the oily phase comprising at least one oil, the weight ratio between the oily phase and the aqueous phase being between 0.75 and 1.25, characterized in that the aqueous phase comprises:
at least one amino acid and/or a salt thereof and/or a derivative thereof,
citric acid and/or a salt thereof, and
salicylic acid, and
the weight ratio between the sum of the contents by weight of citric acid and salicylic acid, and the content by weight of amino acid is between 0.90 and 1.10.

The present invention relates to a two-phase composition consisting of an aqueous phase and an oily phase which are separate, the aqueous phase comprising an amino acid and/or a salt thereof and/or a derivative thereof, citric acid and/or a salt thereof, and salicylic acid. The present invention also relates to the use of such a composition, as well as to a makeup removal method using such a composition.

Compositions consisting of two separate phases, particularly an aqueous phase and an oily phase which are separate and are not emulsified in each other at rest, are generally referred to by the term “two-phase composition.” They differ from emulsions by the fact that, at rest, the two phases are separate instead of being emulsified in each other. The use of these two-phase compositions requires prior stirring in order to form an emulsion, the latter needing to be of sufficient quality and stability to allow for a homogeneous application of both phases on the skin or the keratin material to which it is applied. At rest, said phases must separate quickly from each other and return to their initial state, this phenomenon being more commonly known by the term “phase separation” or “demixing.”

Relatively fast demixing of the two phases after their use is one of the desired qualities of two-phase compositions. Indeed, achieving a rapid phase separation is desirable for various reasons, particularly because a poor separation of the two phases is perceived as unsightly by users and may cause the product to become “three-phase” as it is used overtime.

Two-phase compositions are in particular used for removing makeup from the face, in particular from the eyes. They must therefore have good eye tolerance.

However, the cosmetics market tends to be focused on compositions that are increasingly natural, comprising more environmentally-friendly compounds and/or originating from biomass.

Consequently, there remains a need for new natural two-phase compositions consisting of two separate, non-mixable phases that have both good demixing qualities and good eye tolerance, while still achieving effective makeup removal. “Natural composition” means a cosmetic composition mainly formulated with natural ingredients and/or ingredients of natural origin. “Natural ingredient” means a compound that is obtained directly from the ground or soils or from plants or animals, if applicable, by means of one or more physical processes such as, for example, grinding, refining, distillation, purification, or filtration. Ingredients “of natural origin” means natural compounds having undergone one or more chemical treatments or related industrial treatments bringing about modifications that are not detrimental to the essential qualities of the compound, and/or a compound mainly comprising natural components which may or may not have undergone transformations as indicated above.

The inventors have surprisingly discovered that the combination of an amino acid and/or a salt thereof and/or a derivative thereof, citric acid and/or a salt thereof, and salicylic acid and/or a derivative thereof in the aqueous phase of a two-phase composition, in a weight ratio between the sum of the contents by weight of citric acid and salicylic acid, and the content by weight of amino acid (i.e. the mass ratio of (citric acid+salicylic acid):amino acid) of between 0.90 and 1.10 makes it possible to obtain a two-phase composition that meets these needs. The compositions according to the invention in particular have good eye tolerance and good demixing qualities. The compositions according to the invention are also very efficient for makeup removal. In addition, the compositions according to the invention provide brightness and improve the quality of the keratin materials of the eyes, particularly the eyelashes.

More specifically, the subject matter of the present invention is a cosmetic composition consisting of an aqueous phase and an oily phase which are separate, the oily phase comprising at least one oil, the weight ratio between the oily phase and the aqueous phase being between 0.75 and 1.25, characterized in that the aqueous phase comprises:
at least one amino acid and/or a salt thereof and/or a derivative thereof,
citric acid and/or a salt thereof, and
salicylic acid, and

the weight ratio between the sum of the contents by weight of citric acid and salicylic acid, and the content by weight of amino acid is between 0.90 and 1.10. Such a composition according to the invention is thus a two-phase composition.

The subject matter of the present invention is also a method of makeup removal and/or cleaning and/or care of the keratin materials comprising the application of a composition according to the invention on said keratin materials, in particular the keratin materials of the eyes, preferably the eyelashes.

The subject matter of the present invention is also the use of a composition according to the invention for makeup removal and/or cleaning and/or care of the keratin materials, particularly the keratin materials of the eyes, preferably the eyelashes.

Oily Phase

The oily phase of the compositions according to the invention comprises at least one oil.

Preferably, the oily phase of the composition according to the invention comprises at least one vegetal oil.

The oil or oils of the oily phase are preferably chosen from jojoba seed oil (INCI name: SIMMONDSIA CHINENSIS (JOJOBA) SEED OIL/SIMMONDSIA CHINENSIS SEED OIL), sweet almond oil (INCI name: PRUNUS AMYGDALUS DULCIS (SWEET ALMOND) OIL/PRUNUS DULCIS), refined and deodorized limnanthes alba oil (INCI name: LIMNANTHES ALBA (MEADOWFOAM) SEED OIL/LIMNANTHES ALBA SEED OIL), avocado oil, castor oil, olive oil, sunflower oil, wheat germ oil, sesame oil, peanut oil, grapeseed oil, soybean oil, rapeseed oil, safflower oil, coconut oil, corn oil, hazelnut oil, palm oil, apricot kernel oil, and calophyllum oil and mixtures thereof.

The oily phase can also comprise at least one volatile apolar hydrocarbonated oil, preferably chosen from the hydrocarbon oils having 8 to 28 carbon atoms, and particularly the alkanes branched at C15-C19, such as those marketed by Seppic under the name EMOGREEN L19.

In addition, the oily phase can comprise a fatty acid ester, preferably of natural origin.

As examples of fatty acid esters, let us mention the synthetic esters of fatty acids such as the oils having the formula R₁COOR₂ wherein R₁ represents the residue of a linear or branched higher fatty acid comprising 1 to 40 carbon atoms and R₂ represents a hydrocarbon chain, particularly branched, containing 1 to 40 carbon atoms with R₁+R₂>10, such as for example Purcellin oil (cetostearyl octanoate), isononyl isononanoate, isopropyl myristate, isopropyl palmitate, C12 to C15 alcohol benzoate, hexyl laurate, diisopropyl adipate, 2-ethylhexyl palmitate, octyl-2-dodecyl stearate, octyl-2-dodecyl erucate, isostearyl isostearate, tridecyl trimellitate; octanoates, decanoates or ricinoleates of alcohols or polyalcohols such as propylene glycol dioctanoate; hydroxylated esters, such as isostearyl lactate, octyl hydroxy stearate, octyl dodecyl hydroxy stearate, diisostearyl-malate, triisocetyl citrate; heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate.

Preferably, the fatty acid ester is chosen from isopropyl palmitate, isopropyl myristate, isononyl isononanoate, alkylbenzoate at C12-C15, and mixtures thereof, advantageously the fatty acid ester is isopropyl palmitate.

The oily phase can further comprise conventional cosmetic additives such as scents.

Aqueous Phase

The aqueous phase comprises:
at least one amino acid and/or a salt thereof and/or a derivative thereof,
citric acid and/or a salt thereof, and
salicylic acid and/or a derivative thereof, the weight ratio between the sum of the contents by weight of citric acid and salicylic acid, and the content by weight of amino acid (i.e. The mass ratio of (citric acid+salicylic acid) amino acid) is between 0.90 and 1.10.

Preferably, the weight ratio between the sum of the contents by weight of citric acid and salicylic acid, and the content by weight of amino acid is between 0.92 and 1.05, preferably between 0.95 and 1.02.

Such ratios make it possible to obtain a cosmetic composition having good eye tolerance and at the same time good demixing qualities. These ratio values also impart satisfactory microbiological protection to the composition according to the invention.

Furthermore, the aqueous phase contains water.

The aqueous phase possibly contains at least one organic solvent that is soluble or miscible in water.

The water-soluble or water-miscible solvents suitable for the invention comprise short-chain mono-alcohols for example at C1-C4, such as ethanol, isopropanol; diols or polyols such as glycerol, ethylene glycol, 1,2-propylene glycol, 1,3-butylene glycol, hexylene glycol, diethylene glycol, 1,3-propanediol, 1,2-pentadiol, dipropylene glycol, 2-ethoxyethanol, diethylene glycol monomethylether, triethylene glycol monomethylether, sorbitol, and mixtures thereof. Preferably, the water-soluble or water-miscible organic solvent is 1,3-propanediol.

Preferably, the aqueous phase comprises at least 60% by weight water in relation to the total weight of the aqueous phase, preferably at least 70% by weight, preferably a content of between 80% and 95% by weight.

Amino Acid

The aqueous phase of the composition according to the invention comprises at least one amino acid and/or a salt thereof and/or a derivative thereof.

In this invention, “amino acid derivatives” means amino acids having substitutions either in the carboxyl group of said amino acids or in the amino group carried by the alpha carbon of the carboxyl group, or possibly in both of these groups.

Preferably, the amino acid is arginine or lysine, preferably arginine.

The lysine is preferably L-lysine.

Preferably, the amino acid of the aqueous phase of the cosmetic composition according to the invention is an arginine and/or an organic acid or mineral acid salt thereof and/or a derivative thereof.

The various forms of arginine are generally D-arginine, D,L-arginine, and L-arginine. From the organic acid or mineral acid salts of arginine, let us mention for example the hydrochlorides, glutamates, butyrates, and glycolates.

From the derivatives of L-arginine, let us mention the C1-C4 alkyl esters of L-arginine, with the alkyl being chosen, for example, from methyl, ethyl, propyl, isopropyl, butyl, isobutyl.

Of the derivatives of L-arginine, let us also mention the oligomers of L-arginine (called polyarginine), such as those described in application EP1060739 and in application WO 30 03/072039. Arginine oligomers preferred according to the invention comprise 7 to 15 L-arginine patterns, for example 7, 9, 11, 13, or 15 L-arginine patterns. More specifically, L-arginine will be used.

Preferably, the amino acid is present with a content of between 0.01% and 5% by weight in relation to the total weight of the aqueous phase, preferably between 0.05% and 3% by weight, preferably between 0.1% 2%, advantageously between 0.5% and 1%.

The content of amino acid can also be expressed in relation to the total weight of the composition. Preferably, the amino acid is present with a content of between 0.005% and 3% by weight in relation to the total weight of the composition, preferably between 0.02% and 1.5% by weight, preferably between 0.1% and 1%, advantageously between 0.3% and 0.6%.

Citric Acid

The aqueous phase of the cosmetic composition according to the invention comprises citric acid and/or a salt thereof.

From the citric acid salts, let us mention the salts of citric acid and an alkaline or alkaline-earth metal. Such salts are, in particular, potassium or sodium citrate.

Preferably, the cosmetic composition according to the invention comprises citric acid.

Preferably, the citric acid is present with a content of between 0.01% and 1% by weight in relation to the total weight of the aqueous phase, preferably between 0.05% and 0.8% by weight, preferably between 0.1% 0.7%, advantageously between 0.3% and 0.6%.

The content of citric acid can also be expressed in relation to the total weight of the composition. Preferably, the citric acid is present with a content of between 0.005% and 0.5% by weight in relation to the total weight of the composition, preferably between 0.02% and 0.4% by weight, preferably between 0.05% and 0.35%, advantageously between 0.15% and 0.3%.

Salicylic Acid

The aqueous phase of the composition according to the invention comprises salicylic acid.

Preferably, the salicylic acid is present with a content of between 0.01% and 1% by weight in relation to the total weight of the aqueous phase, preferably between 0.05% and 0.5% by weight, preferably between 0.05% and 0.4%, advantageously between 0.1% and 0.3%.

The content of salicylic acid can also be expressed in relation to the total weight of the composition. Preferably, the salicylic acid is present with a content of between 0.005% and 0.5% by weight in relation to the total weight of the composition, preferably between 0.02% and 0.25% by weight, preferably between 0.02% and 0.2%, advantageously between 0.05% and 0.15%.

Compound According to Formula (I)

The cosmetic composition according to the invention can further comprise at least one compound according to the following formula (I):

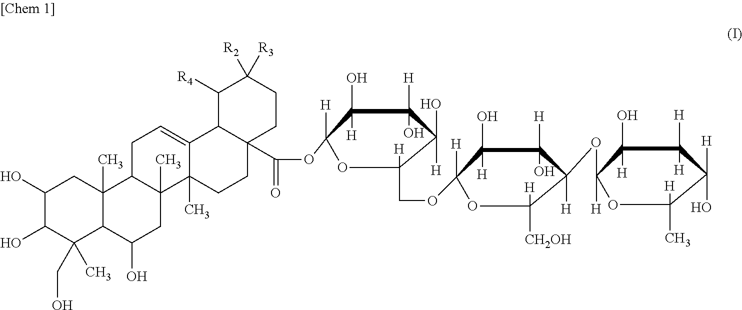

wherein R₁═H or —CH₃; R₂═H or —CH₃; R₃═—CH₃, R₁ and R₂ not being simultaneously H, or an extract of Centella Asiatica comprising a compound or a mixture of compounds according to formula (I).

Preferably, the compound of formula (I) is chosen from madecassoside, terminoloside, and mixtures thereof.

Preferably, the compounds of formula (I) is a mixture of madecassoside and terminoloside comprising a madecassoside content ranging from 30% to 70% by weight in relation to the total weight of the mixture, and especially a content ranging from 45% to 55% by weight.

Preferably, the Centella Asiatica extract comprises more than 95% by weight of a mixture of madecassoside and terminoloside in relation to the weight of the extract.

Preferably, the compound according to formula (I) is present with a content of between 0.001% and 0.5% by weight in relation to the total weight of the aqueous phase, preferably between 0.005% and 0.1% by weight, preferably between 0.01% 0.05%, advantageously between 0.02% and 0.04%.

The content of compound according to formula (I) can also be expressed in relation to the total weight of the composition. Preferably, the compound according to formula (I) is present with a content of between 0.0005% and 0.25% by weight in relation to the total weight of the composition, preferably between 0.002% and 0.05% by weight, preferably between 0.005% 0.02%, advantageously between 0.01% and 0.02%.

Cosmetic Composition

The cosmetic composition according to the invention consists of an aqueous phase and an oily phase which are separate.

Preferably, the weight ratio between the oily phase and the aqueous phase is between 0.75 and 1.25, preferably between 0.78 and 1.22, preferably between 0.80 and 1.10, preferably between 0.81 and 1.

These ratio values make it possible to obtain a cosmetic composition that is excellent at makeup removal and also has a satisfactory visual appearance.

Preferably, the aqueous phase has a pH of between 3.8 and 4.4, preferably between 3.9 and 4.3, and advantageously between 4.0 and 4.2.

Preferably, the aqueous phase is substantially free of monovalent inorganic salt and/or divalent inorganic salt, preferably totally free of monovalent inorganic salt and divalent inorganic salt.

“Substantially free” means that the composition comprises less than 0.1% by weight in relation to the total weight of the composition, preferably less than 0.05% by weight, preferably less than 0.01% by weight of monovalent inorganic salt and/or divalent inorganic salt. Preferably, the composition according to the invention is totally free of monovalent inorganic salt and totally free of divalent inorganic salt.

A monovalent inorganic salt means a salt not comprising any C—H bonds and consisting of a cation and an anion, each carrying a single positive or negative charge, respectively. For example, NaCl is a monovalent salt.

A divalent inorganic salt means a salt not comprising any C—H bonds and consisting of a cation and an anion, each carrying two positive or negative charges, respectively. For example, MgSO₄ is a divalent salt.

Preferably, the cosmetic composition according to the invention is substantially free of surfactant. “Substantially free” means that the composition comprises less than 0.5% by weight in relation to the total weight of the composition, preferably less than 0.3% by weight, preferably less than 0.1% by weight of surfactant. Preferably, the composition according to the invention is totally free of surfactant.

The expressions “between . . . and . . . ”, “ranging from . . . To . . . ”, and “varying from . . . to . . . ” are to be understood as inclusive of the limits, unless specified otherwise.

Concrete, yet non-limiting, examples, illustrating the invention, will now be provided.

Unless stated otherwise, the pressure in the examples is atmospheric pressure.

### EXAMPLES

In all the examples, the compositions are prepared with the ingredients mentioned in the tables hereinbelow, according to the following protocol:

An oily phase is prepared by mixing the ingredients of the oily phase.

The hydrolate is mixed with the arginine in water; the salicylic acid pre-dispersed in the propanediol is then added; the madecassoside is then added; lastly, the pH is adjusted with the citric acid. The aqueous phase is thus obtained.

Lastly, the oily phase is put into contact with the aqueous phase in order to obtain the composition.

In the examples, the ocular tolerance of the compositions was evaluated.

In vivo tests were conducted for that purpose. These are usage tests with consumers (with a minimum of 60 consumers) which consist of a makeup removal routine for 15 days. Observations are made by the consumers themselves, as well as by a dermatologist and an ophthalmologist (toxicological expertise) at the beginning and end of the trial.

In this study, the eye tolerance assessment of the various compositions is indicated by an “OK” or “not OK” result, depending on whether the tolerance is acceptable or not.

Demixing Test Protocol:

Demixing is examined after repeated mixing. The demixing of the two-phase composition must be satisfactory essentially at T24H after mixing:
at ambient temperature after 10 instances of back-and-forth tipping over of the bottle and after 4 successive days of 10 instances of back-and-forth tipping over per day;
After spending 2 months at 45° C., after 10 instances of back-and-forth tipping over of the bottle and after 4 successive days of 10 instances of back-and-forth tipping over per day;
After spending 10 days of −20+20° C. cycles and having undergone 10 instances of back-and-forth tipping over of the bottle and after 4 successive days of 10 instances of back-and-forth tipping over per day.

Microbiological test protocol: this is the test described in detail in ISO standards 11930:2019 and 29621:2017.

### Example 1: Preparation of a Composition According to the Invention, C1, and Comparative Compositions C2*, C3*, and C4*

The composition according to the invention, C1, and comparative compositions C2*, C3*, and C4* are prepared with the ingredients mentioned in the table hereinbelow, according to the protocol described above. The quantities of each ingredient are expressed as a mass ratio in relation to the total weight of the phase (aqueous phase and oily phase) containing it, and not in relation to the total weight of the composition.

TABLE 1

| | | C1 | | | |
|---|---|---|---|---|---|
| | | (according | C2* | C3* | C4* |
| | | to the | (% w/w) | (% w/w) | (% w/w) |
| Phase | Ingredient | invention) | (comparative) | (comparative) | (comparative) |
| Oily | Jojoba seed oil (INCI: | 3.8 | 3.8 | 3.8 | 3.8 |
| phase | SIMMONDSIA | | | | |
| | CHINENSIS | | | | |
| | (JOJOBA) SEED | | | | |
| | OIL/SIMMONDSIA | | | | |
| | CHINENSIS SEED | | | | |
| | OIL) | | | | |
| | Isopropyl palmitate | Qs 100 | Qs 100 | Qs100 | Qs 100 |
| | Sweet almond oil | 18.7 | 18.7 | 10.4 | 18.7 |
| | (INCI: PRUNUS | | | | |
| | AMYGDALUS | | | | |
| | DULCIS (SWEET | | | | |
| | ALMOND) | | | | |
| | OIL/PRUNUS | | | | |
| | DULCIS) | | | | |
| | Refined and | — | — | 9 | — |
| | deodorized | | | | |
| | limnanthes alba oil | | | | |
| | (INCI: LIMNANTHES | | | | |
| | ALBA | | | | |
| | (MEADOWFOAM) | | | | |
| | SEED | | | | |
| | OIL/LIMNANTHES | | | | |
| | ALBA SEED OIL) | | | | |
| | Alkanes branched at | 20 | — | — | 20 |
| | C15-C19 (INCI: | | | | |
| | C15-19 ALKANE) | | | | |
| | Stabilized dicaprylyl | — | 20.8 | 20.8 | — |
| | ether (INCI: | | | | |
| | DICAPRYLYL | | | | |
| | ETHER) | | | | |
| | Perfume | 0.5 | 0.15 | 0.15 | 0.5 |
| Aqueous | Hydrolate | 2 | 2 | 5 | 2 |
| phase | L-Arginine | 0.784 | 0.784 | — | 0.784 |
| | Water | 10 | 10 | — | 10 |
| | Salicylic acid | 0.181 | 0.273 | 0.22 | 0.273 |
| | 1,3-Propanediol | 4.9 | 4.9 | — | — |
| | 1,2-Pentanediol | — | — | 5.5 | 4.9 |
| | Water | Qs 100 | Qs 100 | Qs 100 | Qs 100 |
| | Purified isomers of | 0.03 | 0.03 | — | 0.03 |
| | Centella Asiatica, | | | | |
| | madecassoside/ | | | | |
| | terminoloside mixture, | | | | |
| | purity 95% (INCI: | | | | |
| | MADECASSOSIDE | | | | |
| | Citric acid | 0.58 | 0.6 | — | 0.37 |
| | Sodium chloride | — | — | 2 | — |
| | Magnesium sulfate | — | — | 2 | — |
| | Weight ratio of oily | 0.82 | 0.82 | 0.82 | 0.82 |
| | phase to aqueous | | | | |
| | phase | | | | |
| | Ratio of (citric acid + | 0.97 | 1.11 | — | 0.82 |
| | salicylic acid) to | | | | |
| | arginine | | | | |
| | pH of aqueous phase | 4.1 | 3.6 | 2.7 | 4.5 |
| | Stability (demixing | OK | OK | Not OK | OK |
| | quality) | | | (unclear | |
| | | | | interface due | |
| | | | | to presence | |
| | | | | of small white | |
| | | | | particles) | |
| | Eye tolerance | OK | Not OK | Not OK | OK |
| | Microbiological | OK | OK | OK | Not OK |
| | protection | | | | |

Of these four compositions, only composition C1 according to the invention makes it possible to obtain a composition simultaneously displaying satisfactory demixing, eye tolerance, and microbiological protection.

1. A cosmetic composition consisting of an aqueous phase and an oily phase which are separate, the oily phase comprising at least one oil, the weight ratio between the oily phase and the aqueous phase being between 0.75 and 1.25, characterized in that the aqueous phase comprises:
at least one amino acid and/or a salt thereof and/or a derivative thereof,
citric acid and/or a salt thereof, and
salicylic acid, and
the weight ratio between the sum of the contents by weight of citric acid and salicylic acid, and the content by weight of amino acid is between 0.90 and 1.10.

2. The cosmetic composition according to claim 1, wherein the amino acid is an arginine and/or an organic acid or mineral acid salt thereof and/or a derivative thereof.

3. The cosmetic composition according to claim 1, wherein the amino acid is present with a content of between 0.01% and 5% by weight in relation to the total weight of the aqueous phase.

4. The composition according to claim 1, wherein the citric acid is present with a content of between 0.01% and 1% by weight in relation to the total weight of the aqueous phase.

5. The composition according to claim 1, wherein the salicylic acid is present with a content of between 0.01% and 1% by weight in relation to the total weight of the aqueous phase.

6. The composition according to claim 1, further comprising at least one compound according to the following formula (I): 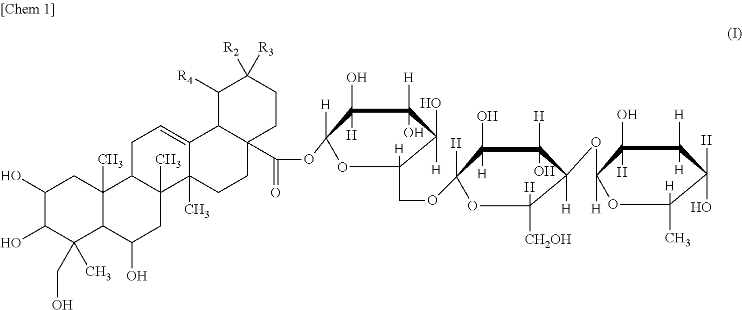
wherein R₁═H or —CH₃; R₂═H or —CH₃; R₃═—CH₃, R₁ and R₂ not being simultaneously H, or an extract of Centella Asiatica comprising a compound or a mixture of compounds according to formula (I).

7. The composition according to claim 6, wherein the compound of formula (I) is chosen from madecassoside, terminoloside, and mixtures thereof.

8. The composition according to claim 6, wherein the compound according to formula (I) is present with a content of between 0.001% and 0.5% by weight in relation to the total weight of the aqueous phase.

9. The composition according to claim 1, wherein the aqueous phase has a pH of between 3.8 and 4.4.

10. The composition according to claim 1, wherein the aqueous phase is substantially free of monovalent inorganic salt and/or divalent inorganic salt.

11. A method of makeup removal and/or cleaning and/or care of the keratin materials comprising the application of a composition according to claim 1 on said keratin materials.

12. (canceled)

13. The cosmetic composition according to claim 2, wherein the amino acid is present with a content of between 0.01% and 5% by weight in relation to the total weight of the aqueous phase.

14. The composition according claim 2, wherein the citric acid is present with a content of between 0.01% and 1% by weight in relation to the total weight of the aqueous phase.

15. The composition according claim 3, wherein the citric acid is present with a content of between 0.01% and 1% by weight in relation to the total weight of the aqueous phase.

16. The composition according to claim 2, wherein the salicylic acid is present with a content of between 0.01% and 1% by weight in relation to the total weight of the aqueous phase.

17. The composition according to claim 3, wherein the salicylic acid is present with a content of between 0.01% and 1% by weight in relation to the total weight of the aqueous phase.

18. The composition according to claim 4, wherein the salicylic acid is present with a content of between 0.01% and 1% by weight in relation to the total weight of the aqueous phase.

19. The composition according to claim 2, further comprising at least one compound according to the following formula (I): 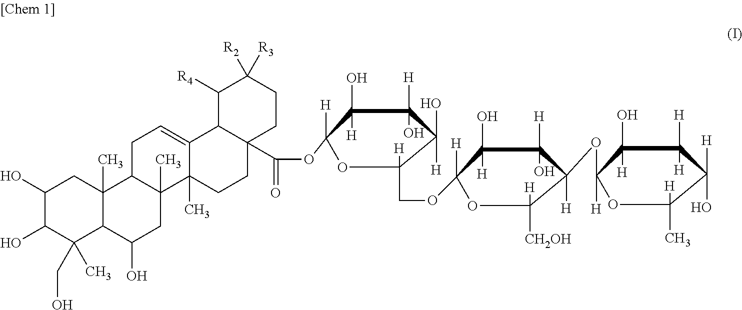
wherein R₁═H or —CH₃; R₂═H or —CH₃; R₃═—CH₃, R₁ and R₂ not being simultaneously H, or an extract of Centella Asiatica comprising a compound or a mixture of compounds according to formula (I).

20. The composition according to claim 3, further comprising at least one compound according to the following formula (I): 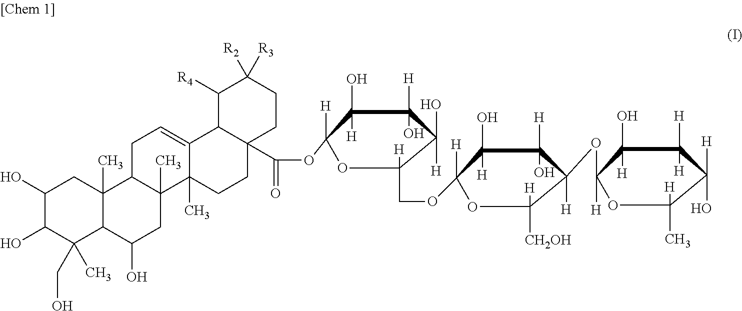
wherein R₁═H or —CH₃; R₂═H or —CH₃; R₃═—CH₃, R₁ and R₂ not being simultaneously H, or an extract of Centella Asiatica comprising a compound or a mixture of compounds according to formula (I).

21. The composition according to claim 4, further comprising at least one compound according to the following formula (I): 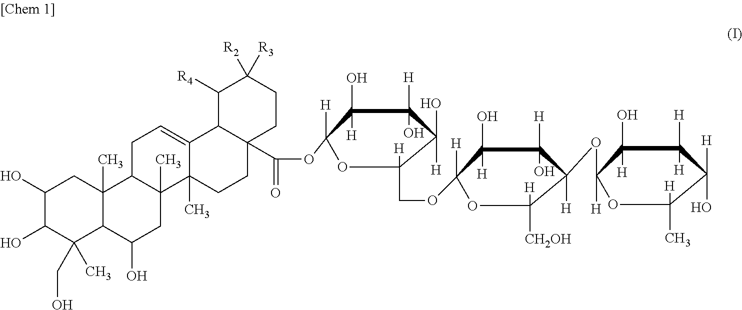
wherein R₁═H or —CH₃; R₂═H or —CH₃; R₃═—CH₃, R₁ and R₂ not being simultaneously H, or an extract of Centella Asiatica comprising a compound or a mixture of compounds according to formula (I).
